(19) United States
(12) Patent Application Publication
(10) Pub. No.: US 2005/0267145 A1
(43) Pub. Date: Dec. 1, 2005

(54) Treatment for lung cancer

(76) Inventors: Bryon A. Merrill, River Falls, WI (US); Paul B. Myrdal, Tucson, AZ (US); Paul D. Wightman, Woodbury, MN (US)

Correspondence Address: 3M INNOVATIVE PROPERTIES COMPANY, PO BOX 33427, ST. PAUL, MN, 55133-3427, US

(21) Appl. No.: 11/141,655

(22) Filed: May 31, 2005

Related U.S. Application Data
(60) Provisional application No. 60/575,496, filed on May 28, 2004.

(51) Int. Cl. A61K 31/4745; A61K 31/19

(52) U.S. Cl. 514/292; 514/575

(57) ABSTRACT

The present invention provides methods, pharmaceutical compositions, and pharmaceutical combinations useful for treating lung cancer. Generally, the compositions include a 5-LO inhibitor in an amount effective to inhibit 5-lipoxygenase in an inhalable formulation. In some cases, the formulation may further include an IRM compound. Generally, the pharmaceutical combinations include a 5-LO inhibitor and an IRM compound in an inhalable formulation. Generally, the methods include administering to the subject an inhalable formulation that comprises a 5-lipoxygenase inhibitor having a cLogP of at least about 4.0 in an amount effective for treating lung cancer.

### CROSS REFERENCE TO RELATED APPLICATION

This application claims the benefit of U.S. Provisional Patent Application No. 60/575,496, filed May 28, 2004.

### BACKGROUND

Cancer of the lung is the most prevalent cause of cancer-related deaths in the United States. American Cancer Society statistics for the year 2002 attribute 154,900 deaths to lung cancer and indicate that 169,400 new cases were diagnosed in that year. The five-year survival rate for lung cancer patients is 15%, well below the average five-year survival rate for all cancers, 62%. The low survival rate for lung cancer illustrates the aggressive and lethal nature of lung cancer relative to other forms of cancer. Current therapeutic strategies have had minimal effect on the lung cancer mortality rate.

Leukotrienes are potent inflammatory mediators in asthma and contribute to increased mucus production, bronchoconstriction, and eosinophil infiltration. An initial event in asthma appears to be the release of inflammatory mediators including, e.g., leukotrienes, triggered by exposure to allergens, irritants, cold air, or exercise. Release of the inflammatory mediators turns on the cellular responses that lead to the asthma reaction: contraction of the airway muscles, swelling of the airway lining, and flooding of the remaining airway space with sticky mucus. Leukotrienes are produced via the lipoxygenase pathway of arachidonic acid metabolism by mast cells, eosinophils and alveolar macrophages. One enzyme in the lipoxygenase pathway is 5-lipoxygenase (5-LO). Thus, 5-lipoxygenase inhibitors have been used as treatments for asthma.

The 5-LO pathway has also recently received attention from the National Cancer Institute and academic and industrial laboratories as a chemotherapeutic agent for a variety of cancers because it has been proposed that the 5-LO pathway also may be involved in cellular growth and proliferation. 5-LO inhibitors have been examined as a potential chemotherapeutic agent for treating a variety of cancers such as, for example, prostate, colon, breast, pancreatic, and lung. The 5-LO inhibitor zileuton has been shown effective at preventing lung tumors and slowing the growth and progression of adenoma to carcinoma in mice when administered orally.

An ongoing need exists to find additional compounds and pharmaceutical combinations that may be effective for treating lung cancer.

### SUMMARY

It has been found that additional inhibitors of the 5-LO pathway may be effective for treating lung cancer. Additionally, it has been found that a 5-LO inhibitor may be effective for treating lung cancer when delivered in an inhalable formulation.

Accordingly, the present invention provides an inhalable pharmaceutical composition that includes a 5-LO inhibitor having a cLogP of at least 4.0 in an amount effective to inhibit 5-lipoxygenase. In some embodiments, the composition may further include one or more additives such as, for example, an IRM compound.

In another aspect, the present invention also provides pharmaceutical combination that includes a 5-lipoxygenase inhibitor in an inhalable formulation in an amount effective to inhibit 5-lipoxygenase and an effective amount of an IRM compound. In some embodiments, the pharmaceutical combination may be provided in two or more formulations. In some cases, the IRM compound may be provided in a formulation other than an inhalable formulation.

In another aspect, the invention provides a method of treating lung cancer. Generally, the method includes administering to a subject an inhalable formulation that comprises a 5-lipoxygenase inhibitor having a cLogP of at least about 4.0 in an amount effective for treating lung cancer. In some embodiments, the method further includes administering to the subject an IRM compound in an amount effective, in combination with the 5-LO inhibitor, to treat lung cancer.

Various other features and advantages of the present invention should become readily apparent with reference to the following detailed description, examples, claims and appended drawings. In several places throughout the specification, guidance is provided through lists of examples. In each instance, the recited list serves only as a representative group and should not be interpreted as an exclusive list.

### Brief Description of the Drawings

FIG. 1 is a bar graph showing adenoma counts in mice treated with various 5-lipoxygenase inhibitors.

FIG. 2 is a line graph showing that zymosan-induced production of LTC₄ and PGE2 can be inhibited by an immune response modifier (IRM) compound.

### DETAILED DESCRIPTION OF ILLUSTRATIVE EMBODIMENTS OF THE INVENTION

The present invention provides compounds, pharmaceutical combinations, and methods effective for treating lung cancer.

While there may be identifiable molecular targets for the chemoprevention of lung cancer, a therapeutic agent cannot be effective if it does not reach the target site or is not presented at sufficient concentration. Inhalation drug delivery offers the advantage of avoiding first pass metabolism while directly targeting the lung. Because of high serum binding and first pass metabolism, an unacceptably high systemic dose of a 5-LO inhibitor may be required to achieve inhibition of 5-LO—and thus, therapeutic treatment—in the lung. In contrast, relatively high levels of a 5-LO inhibitor may be delivered by inhalation directly into the lung, thereby increasing efficacy of the therapy while at the same time decreasing systemic exposure.

In one aspect, the invention provides certain 5-LO inhibitors that are useful for treating lung cancer when delivered by inhalation. “Treat” and variations thereof (e.g., “treating” or “treatment”) can refer to prophylactic (i.e., preventative) treatment, therapeutic treatment, or both. Thus, treating lung cancer can include, for example, administering a 5-LO inhibitor before any sign or symptom of lung cancer is detected in order to prevent or reduce the likelihood that a subject may develop lung cancer. Alternatively, treating lung cancer can include, for example, administering a 5-LO inhibitor to a subject diagnosed as having lung cancer in order to ameliorate one or more signs or symptoms of the disease.

Unless otherwise indicated, reference to a compound can include the compound in any pharmaceutically acceptable form, including any isomer (e.g., diastereomer or enantiomer), salt, solvate, polymorph, and the like. In particular, if a compound is optically active, reference to the compound can include each of the compound's enantiomers as well as racemic mixtures of the enantiomers.

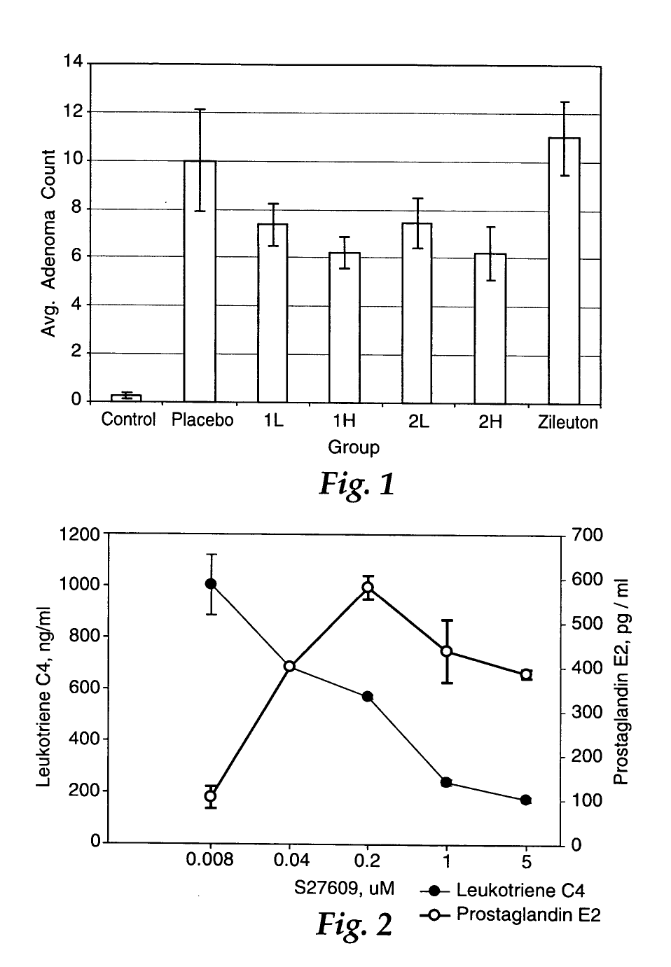

FIG. 1 shows the effectiveness of inhaled 5-LO inhibitors for decreasing pulmonary adenoma counts in mice after exposure to a carcinogen. The pulmonary adenoma counts for the inhalation-delivered 5-LO inhibitors (Groups 1L, 1H, 2L, and 2H) represented up to a 40% reduction in the number of pulmonary adenomas compared to the placebo group. In contrast, orally-administered zileuton, which has been shown to reduce the number of lung tumors when provided at high doses, did not have any apparent effect on the number of pulmonary adenomas despite being administered at doses in excess of 500-fold greater than the compounds administered via nose-only inhalation.

One desirable property of a 5-LO inhibitor to be administered by inhalation is that its duration of action in the lung be of sufficient length to minimize the number of treatments required per day. The ability of a compound to remain associated with the tissue or cell bearing its molecular target could provide an extended duration of action in vivo. This property was initially assessed in mouse macrophages. Compounds were initially evaluated at a single concentration of 10 μM. Each of the compounds was also active in the rat lung in situ assay. Results are shown in Table 2.

Inhibition of leukotriene formation is not merely a matter of potency of the compound in inhibiting the 5-lipoxygenase enzyme, however. The two compounds that show the highest sustained activity following washout (see Table 2) are also the most lipophilic of this group, as demonstrated by their calculated LogP (cLogP) values. LogP value, which is the logarithm of a compound's partition coefficient between n-octanol and water, is a well-established measure of lipophilicity. The LogP value of a hydrophobic compound is greater than the LogP value of a hydrophilic compound. The calculated LogP (cLogP) may be computed using commercially available software. As used herein, cLogP refers to LogP values calculated using CLOGP, v.4.2, including version 22 of the fragment database, provided with SYBYL 6.9.1 (Tripos, Inc. St. Louis, Mo.).

Thus in one aspect, the invention provides a method for treating lung cancer in a subject. Generally, the method includes administering to the subject a 5-lipoxygenase inhibitor that has a cLogP value of at least about 4.0 in an inhalable formulation in an amount effective for treating lung cancer. In some embodiments, the 5-LO inhibitor can have a cLogP value of at least about 5.0. In other embodiments, the 5-LO inhibitor can have a cLogP value of at least about 5.4. In still other embodiments, the 5-LO inhibitor can have a cLogP value of at least about 5.6 such as, for example, a cLogP value of at least about 6.6.

The compound may be provided in any formulation suitable for administration to a subject. Suitable types of formulations are described, for example, in U.S. Pat. Nos. 5,225,183; 5,776,432; 6,315,985; 5,569,450; 6,518,239; 6,309,623; and International Patent Publication No. WO 03/86350. The compound may be provided in any suitable form including but not limited to a solution, a suspension, an emulsion, or any form of mixture. The compound may be delivered in formulation with any pharmaceutically acceptable excipient, carrier, or vehicle. For example, the formulation may include one or more propellants, cosolvents, or other additives.

Suitable propellants include, for example, hydrochlorofluorocarbons (HFCs), such as 1,1,1,2-tetrafluoroethane (also referred to as propellant 134a, HFC-134a, or HFA-134a) and 1,1,1,2,3,3,3-heptafluoropropane (also referred to as propellant 227, HFC-227, or HFA-227), carbon dioxide, dimethyl ether, butane, propane, or mixtures thereof.

A formulation can include an optional cosolvent—or a mixture of cosolvents—such as, for example, ethanol or isopropanol.

Other additives, such as adjuvants, lubricants, surfactants, bulking agents, and taste masking ingredients, can also be included. In some embodiments, the formulation may include an immune response modifier (IRM) compound. Suitable IRM compounds are described in detail below.

Examples of suitable surfactants include: oils derived from natural sources, such as, corn oil, olive oil, cotton seed oil and sunflower seed oil, sorbitan trioleate, sorbitan monooleate, sorbitan monolaurate, polyoxyethylene sorbitan monolaurate, polyoxyethylene sorbitan monooleate, lecithins, oleyl polyoxyethylene ether, stearyl polyoxyethylene, lauryl polyoxyethylene ether, oleyl polyoxyethylene ether, Block copolymers of oxyethylene and oxypropylene, oleic acid, diethylene glycol dioleate, tetrahydrofurfuryl oleate, ethyl oleate, isopropyl myristate, glyceryl trioleate, glyceryl monolaurate, glyceryl mono-oleate, glyceryl monostearate, glyceryl monoricinoleate, cetyl alcohol, stearyl alcohol, polyethylene glycol 400, and cetyl pyridinium chloride.

Examples of suitable bulking agents include lactose, DL-alanine, ascorbic acid, glucose, sucrose, D(+)trehalose, D-galactose, maltose, D(+)raffinose pentahydrate, sodium saccharin, polysaccharides, such as starches, modified celluloses, dextrins or dextrans, other amino acids, such as glycine, salts, such as sodium chloride, calcium carbonate, sodium tartrate, or calcium lactate.

The composition of a formulation suitable for practicing the invention will vary according to factors known in the art including but not limited to the physical and chemical nature of the 5-LO inhibitor, the nature of the carrier, the intended dosing regimen, the presence and identity of any additives such as, for example, an IRM compound, and the species to which the formulation is being administered. Accordingly, it is not practical to set forth generally the composition of a formulation effective for treating lung cancer for all possible applications. Those of ordinary skill in the art, however, can readily determine an appropriate formulation with due consideration of such factors.

In some embodiments, the methods of the present invention include administering a 5-LO inhibitor to a subject in a formulation of, for example, from about 0.0001% to about 10% (unless otherwise indicated, all percentages provided herein are weight/weight with respect to the total formulation) to the subject, although in some embodiments the 5-LO inhibitor may be administered using a formulation that provides 5-LO inhibitor in a concentration outside of this range. In certain embodiments, the method includes administering to a subject a formulation that includes from about 0.01% to about 5% 5-LO inhibitor, for example, a formulation that includes about 5% 5-LO inhibitor.

An amount of a 5-LO inhibitor effective for treating lung cancer is an amount sufficient to ameliorate at least one sign or symptom of lung cancer. An effective amount of a 5-LO inhibitor may, for example, decrease the subject's likelihood of developing a tumor, decrease the number and/or size of tumors, may slow the growth of tumors, or increase the subject's five-year survival likelihood. The precise amount of 5-LO inhibitor effective for treating lung cancer will vary according to factors known in the art including but not limited to the physical and chemical nature of the 5-LO inhibitor, the nature of the carrier, the intended dosing regimen, the presence and identity of any additives such as, for example, an IRM compound, and the species to which the formulation is being administered. Accordingly, it is not practical to set forth generally the amount that constitutes an amount of 5-LO inhibitor effective for treating lung cancer for all possible applications. Those of ordinary skill in the art, however, can readily determine the appropriate amount with due consideration of such factors.

In some embodiments, the methods of the present invention include administering sufficient 5-LO inhibitor to provide a dose of, for example, from about 10 μg/kg to about 100 mg/kg, although in some embodiments the methods of the present invention may be performed by administering the 5-LO inhibitor at a dose outside this range. In certain embodiments, the dose of 5-LO inhibitor may be at least about 50 μg/kg to about 10 mg/kg. In one particular embodiment, the dose of 5-LO inhibitor may be about 60 μg/kg. In another embodiment, the dose of 5-LO inhibitor may be about 80 μg/kg. In another embodiment, the dose of 5-LO inhibitor may be about 220μg/kg. In another embodiment, the dose of 5-LO inhibitor may be about 425 μg/kg. In another embodiment, the dose of 5-LO inhibitor may be about 800 μg/kg. In yet another embodiment, the dose of 5-LO inhibitor may be about 1 mg/kg.

The dosing regimen may depend at least in part on many factors known in the art including but not limited to the physical and chemical nature of the 5-LO inhibitor, the nature of the carrier, the dose of 5-LO inhibitor being administered, the presence and identity of any additives such as, for example, an IRM compound, and the species to which the formulation is being administered. Accordingly it is not practical to set forth generally the dosing regimen effective for treating lung cancer for all possible applications. Those of ordinary skill in the art, however, can readily determine an appropriate dosing regimen with due consideration of such factors.

In some embodiments of the invention, the 5-LO inhibitor may be administered, for example, from about once per year to multiple administrations per day, although in some embodiments the methods of the invention may be performed by administering the 5-LO inhibitor at a frequency outside this range. For example, 5-LO inhibitor may be administered to a subject at a frequency of about three times per year to about once per day. Thus, the 5-LO inhibitor may be administered about once every four months, once every two months, once every six weeks, or once per month. In other embodiments, the 5-LO inhibitor may be administered at least once per week such as, for example, once per day, five days per week. In other embodiments, the 5-LO inhibitor may be administered once per day.

In some cases, the dosing regiment may include a repeated dosing cycle that specifies a certain number of doses over a defined period of time followed by a period in which the 5-LO inhibitor is not administered. For example, a dosing cycle may include five days per week of treatment and two days per week in which no 5-LO inhibitor is administered.

The 5-LO inhibitor may be administered for any period necessary to achieve the desired level of treatment. For example, treatment may continue until signs or symptoms of a tumor are slowed, reduced, ameliorated, or reversed to a desired extent. In some embodiments, a desired level of treatment may include slowing the growth rate of an existing tumor, reducing the size or number of tumors, or even clearing the subject of tumor cells. In cases in which the 5-LO inhibitor is administered prophylactically, treatment may continue until the likelihood that the subject will develop a tumor is reduced to a desired extent.

In some embodiments, the 5-LO inhibitor is administered to a subject over a period that can range from about one week to about two years, although some embodiments of the methods of the invention may be performed by administering the 5-LO inhibitor for a period outside this range. In some embodiments, the 5-LO inhibitor may be administered over a period of from about one month to about six months, for example, for a period of about sixteen weeks. When the dosing regimen includes a repeated dosing cycle, the duration of the treatment may include a specified number of dosing cycles. For example, treatment may be specified for six, eight, or 16 one-week dosing cycles.

As noted above, in certain embodiments of the invention, the 5-LO formulation may include an immune response modifier (IRM) compound. Table 3 shows that each of a 5-LO inhibitor (zileuton) and an IRM compound inhibits tumor growth and may be combined to provide even more effective tumor inhibition.

IRMs include compounds that possess potent immunomodulating activity including but not limited to antiviral and antitumor activity. Certain IRMs modulate the production and secretion of cytokines. For example, certain IRM compounds induce the production and secretion of cytokines such as, e.g., Type I interferons, TNF-α, IL-1, IL-6, IL-8, IL-10, IL-12, MIP-1, and/or MCP-1.

Certain IRMs also may demonstrate measurable 5-LO inhibitory activity (FIG. 2). Consequently, an IRM compound may be useful administered in combination with a 5-LO inhibitor—either to augment the inhibition of 5-LO and/or to stimulate an immune response against cells of the tumor. A tumor-specific immune response may be stimulated if the combination includes a 5-LO inhibitor, an IRM compound, and a tumor-specific antigen. Therapeutic combinations that include IRM compounds, and methods of raising antigen-specific immune responses are described, for example, in U.S. Pat. No. 6,083,505, U.S. Patent Publication Nos. US2004/0014779 and US 2004/0091491, and U.S. patent application Ser. Nos. 10/748,010 and 10/777,310.

Certain IRMs are small organic molecules (e.g., molecular weight under about 1000 Daltons, preferably under about 500 Daltons, as opposed to large biological molecules such as proteins, peptides, and the like) such as those disclosed in, for example, U.S. Pat. Nos. 4,689,338; 4,929,624; 5,266,575; 5,268,376; 5,346,905; 5,352,784; 5,389,640; 5,446,153; 5,482,936; 5,756,747; 6,110,929; 6,194,425; 6,331,539; 6,376,669; 6,451,810; 6,525,064; 6,541,485; 6,545,016; 6,545,017; 6,573,273; 6,656,938; 6,660,735; 6,660,747; 6,664,260; 6,664,264; 6,664,265; 6,667,312; 6,670,372; 6,677,347; 6,677,348; 6,677,349; 6,683,088; 6,756,382; 6,797,718; and 6,818,650; U.S. Patent Publication Nos. 2004/0091491; 2004/0147543; and 2004/0176367; and International Publication Nos. WO 2005/18551, WO 2005/18556, and WO 2005/20999.

Additional examples of small molecule IRMs include certain purine derivatives (such as those described in U.S. Pat. Nos. 6,376,501, and 6,028,076), certain imidazoquinoline amide derivatives (such as those described in U.S. Pat. No. 6,069,149), certain imidazopyridine derivatives (such as those described in U.S. Pat. No. 6,518,265), certain benzimidazole derivatives (such as those described in U.S. Pat. No. 6,387,938), certain derivatives of a 4-aminopyrimidine fused to a five membered nitrogen containing heterocyclic ring (such as adenine derivatives described in U.S. Pat. Nos. 6,376,501; 6,028,076 and 6,329,381; and in WO 02/08905), and certain 3-βD-ribofuranosylthiazolo[4,5-d]pyrimidine derivatives (such as those described in U.S. Publication No. 2003/0199461).

Other IRMs include large biological molecules such as oligonucleotide sequences. Some IRM oligonucleotide sequences contain cytosine-guanine dinucleotides (CpG) and are described, for example, in U.S. Pat. Nos. 6,194,388; 6,207,646; 6,239,116; 6,339,068; and 6,406,705. Some CpG-containing oligonucleotides can include synthetic immunomodulatory structural motifs such as those described, for example, in U.S. Pat. Nos. 6,426,334 and 6,476,000. Other IRM nucleotide sequences lack CpG sequences and are described, for example, in International Patent Publication No. WO 00/75304.

Other IRMs include biological molecules such as aminoalkyl glucosaminide phosphates (AGPs) and are described, for example, in U.S. Pat. Nos. 6,113,918; 6,303,347; 6,525,028; and 6,649,172.

In some embodiments, suitable IRM compounds include but are not limited to the small molecule IRM compounds described above. Suitable small molecule IRM compounds include, for example, imidazoquinoline amines including but not limited to substituted imidazoquinoline amines such as, for example, amide substituted imidazoquinoline amines, sulfonamide substituted imidazoquinoline amines, urea substituted imidazoquinoline amines, aryl ether substituted imidazoquinoline amines, heterocyclic ether substituted imidazoquinoline amines, amido ether substituted imidazoquinoline amines, sulfonamido ether substituted imidazoquinoline amines, urea substituted imidazoquinoline ethers, thioether substituted imidazoquinoline amines, hydroxylamine substituted imidazoquinoline amines, oxime substituted imidazoquinoline amines, 6-, 7-, 8-, or 9-aryl, heteroaryl, aryloxy or arylalkyleneoxy substituted imidazoquinoline amines, and imidazoquinoline diamines; tetrahydroimidazoquinoline amines including but not limited to amide substituted tetrahydroimidazoquinoline amines, sulfonamide substituted tetrahydroimidazoquinoline amines, urea substituted tetrahydroimidazoquinoline amines, aryl ether substituted tetrahydroimidazoquinoline amines, heterocyclic ether substituted tetrahydroimidazoquinoline amines, amido ether substituted tetrahydroimidazoquinoline amines, sulfonamido ether substituted tetrahydroimidazoquinoline amines, urea substituted tetrahydroimidazoquinoline ethers, thioether substituted tetrahydroimidazoquinoline amines, hydroxylamine substituted tetrahydroimidazoquinoline amines, oxime substituted tetrahydroimidazoquinoline amines, and tetrahydroimidazoquinoline diamines; imidazopyridine amines including but not limited to amide substituted imidazopyridine amines, sulfonamide substituted imidazopyridine amines, urea substituted imidazopyridine amines, aryl ether substituted imidazopyridine amines, heterocyclic ether substituted imidazopyridine amines, amido ether substituted imidazopyridine amines, sulfonamido ether substituted imidazopyridine amines, urea substituted imidazopyridine ethers, and thioether substituted imidazopyridine amines; 1,2-bridged imidazoquinoline amines; 6,7-fused cycloalkylimidazopyridine amines; imidazonaphthyridine amines; tetrahydroimidazonaphthyridine amines; oxazoloquinoline amines; thiazoloquinoline amines; oxazolopyridine amines; thiazolopyridine amines; oxazolonaphthyridine amines; thiazolonaphthyridine amines; pyrazolopyridine amines; pyrazoloquinoline amines; tetrahydropyrazoloquinoline amines; pyrazolonaphthyridine amines; tetrahydropyrazolonaphthyridine amines; and 1H-imidazo dimers fused to pyridine amines, quinoline amines, tetrahydroquinoline amines, naphthyridine amines, or tetrahydronaphthyridine amines.

In certain embodiments, the IRM compound may be an imidazoquinoline amine such as, for example, 4-amino-α,α,2-trimethyl-1H-imidazo[4,5-c]quinoline-1-ethanol or 4-amino-α,α-dimethyl-2-ethoxymethyl-1H-imidazo[4,5-c]quinolin-1-ethanol.

In certain other embodiments, the IRM compound may be an imidazonaphthyridine amine, a tetrahydroimidazonaphthyridine amine, an oxazoloquinoline amine, a thiazoloquinoline amine, an oxazolopyridine amine, a thiazolopyridine amine, an oxazolonaphthyridine amine, a thiazolonaphthyridine amine, a pyrazolopyridine amine, a pyrazoloquinoline amine, a tetrahydropyrazoloquinoline amine, a pyrazolonaphthyridine amine, or a tetrahydropyrazolonaphthyridine amine.

In certain embodiments, the IRM compound may be a substituted imidazoquinoline amine, a tetrahydroimidazoquinoline amine, an imidazopyridine amine, a 1,2-bridged imidazoquinoline amine, a 6,7-fused cycloalkylimidazopyridine amine, an imidazonaphthyridine amine, a tetrahydroimidazonaphthyridine amine, an oxazoloquinoline amine, a thiazoloquinoline amine, an oxazolopyridine amine, a thiazolopyridine amine, an oxazolonaphthyridine amine, a thiazolonaphthyridine amine, a pyrazolopyridine amine, a pyrazoloquinoline amine, a tetrahydropyrazoloquinoline amine, a pyrazolonaphthyridine amine, or a tetrahydropyrazolonaphthyridine amine.

As used herein, a substituted imidazoquinoline amine refers to an amide substituted imidazoquinoline amine, a sulfonamide substituted imidazoquinoline amine, a urea substituted imidazoquinoline amine, an aryl ether substituted imidazoquinoline amine, a heterocyclic ether substituted imidazoquinoline amine, an amido ether substituted imidazoquinoline amine, a sulfonamido ether substituted imidazoquinoline amine, a urea substituted imidazoquinoline ether, a thioether substituted imidazoquinoline amine, a hydroxylamine substituted imidazoquinoline amine, an oxime substituted imidazoquinoline amine, a 6-, 7-, 8-, or 9-aryl, heteroaryl, aryloxy or arylalkyleneoxy substituted imidazoquinoline amine, or an imidazoquinoline diamine. As used herein, substituted imidazoquinoline amines specifically and expressly exclude 1-(2-methylpropyl)-1H-imidazo[4,5-c]quinolin-4-amine and 4-amino-α,α-dimethyl-2-ethoxymethyl-1H-imidazo[4,5-c]quinolin-1-ethanol.

In some cases, the IRM compound may be provided in the same formulation as the 5-LO inhibitor. In other cases, the IRM compound may be provided in a separate formulation. Suitable formulations for administering the IRM compound include the inhalable formulations described above for administering the 5-LO inhibitor. Additionally, suitable formulations for administering the IRM compound include those described, for example, in U.S. Pat. No. 5,736,553; U.S. Pat. No. 5,238,944; U.S. Pat. No. 5,939,090; U.S. Pat. No. 6,365,166; U.S. Pat. No. 6,245,776; U.S. Pat. No. 6,486,168; European Patent No. EP 0 394 026; and U.S. Patent Publication No. 2003/0199538. The IRM compound may be provided in any suitable form including but not limited to a solution, a suspension, an emulsion, or any form of mixture. The IRM compound may be delivered in formulation with any pharmaceutically acceptable excipient, carrier, or vehicle. For example, the formulation may be delivered in a conventional topical dosage form such as, for example, a cream, an ointment, an aerosol formulation, a non-aerosol spray, a gel, a lotion, and the like. The formulation may further include one or more additives including but not limited to adjuvants, skin penetration enhancers, colorants, fragrances, flavorings, moisturizers, thickeners, and the like.

In some embodiments, the methods of the present invention include administering IRM to a subject in a formulation of, for example, from about 0.0001% to about 10%, although in some embodiments the IRM compound may be administered using a formulation that provides IRM compound in a concentration outside of this range. In certain embodiments, the method includes administering to a subject a formulation that includes from about 0.01% to about 5% IRM compound, for example, a formulation that includes from about 0.1% to about 5% IRM compound.

An amount of an IRM compound effective for treating lung cancer is an amount sufficient, in combination with a 5-LO inhibitor, to ameliorate at least one sign or symptom of lung cancer. An effective amount of an IRM compound may, for example, decrease the subject's likelihood of developing a tumor, decrease the number and/or size of tumors, may slow the growth of tumors, or increase the subject's five-year survival likelihood. The precise amount of IRM compound effective for treating lung cancer will vary according to factors known in the art including but not limited to the physical and chemical nature of the IRM compound, the identity and potency of the 5-LO inhibitor, the nature of the carrier, the intended dosing regimen, the state of the subject's immune system (e.g., suppressed, compromised, stimulated), and the species to which the formulation is being administered. Accordingly, it is not practical to set forth generally the amount that constitutes an amount of IRM compound effective for treating lung cancer for all possible applications. Those of ordinary skill in the art, however, can readily determine the appropriate amount with due consideration of such factors.

In some embodiments, the methods of the invention include administering sufficient IRM compound to provide a dose of, for example, from about 100 ng/kg to about 50 mg/kg to the subject, although in some embodiments the methods may be performed by administering IRM compound in a dose outside this range. In some of these embodiments, the method includes administering sufficient IRM compound to provide a dose of from about 1 μg/kg to about 5 mg/kg to the subject, for example, a dose of from about 10 μg/kg to about 1 mg/kg.

The dosing regimen may depend at least in part on many factors known in the art including but not limited to the physical and chemical nature of the IRM compound, the identity and potency of the 5-LO inhibitor, the nature of the carrier, the amount of IRM being administered, the state of the subject's immune system (e.g., suppressed, compromised, stimulated), and the species to which the formulation is being administered. Accordingly it is not practical to set forth generally the dosing regimen effective for treating lung cancer for all possible applications. Those of ordinary skill in the art, however, can readily determine an appropriate dosing regimen with due consideration of such factors.

In certain embodiments, the formulation may include both the 5-LO inhibitor and the IRM compound. In such cases, the dosing regimen for the IRM compound will be the same as the dosing regimen of the 5-LO inhibitor. In other cases, however, the 5-LO inhibitor and the IRM compound may be provided in separate formulations. In such cases, the dosing regimen for the IRM compound may be the same as, similar to, or different than the dosing regimen for the 5-LO inhibitor.

In some embodiments of the invention, the IRM compound may be administered, for example, from about once per month to multiple administrations per day, although in some embodiments the methods of the invention may be performed by administering the IRM compound at a frequency outside this range. For example, IRM compound may be administered to a subject at a frequency of about once per week to about once per day. Thus, in some embodiments, the WRM compound may be administered to the subject once per day, five days per week. In other embodiments, the IRM compound may be administered once per day.

In some cases, the dosing regimen may include a repeated dosing cycle that specifies a certain number of doses over a defined period of time followed by a period in which the IRM compound is not administered. For example, a dosing cycle may include five days per week of treatment and two days per week in which no IRM compound is administered.

The IRM compound may be administered for as long as desired to achieve the desired level of treatment. For example, treatment may continue until signs or symptoms of a tumor are slowed, reduced, ameliorated, or reversed to a desired extent. In some embodiments, a desired level of treatment may include slowing the growth rate of an existing tumor, reducing the size or number of tumors, or even clearing the subject of tumor cells. In cases in which the IRM compound is administered prophylactically, treatment may continue until the likelihood that the subject will develop a tumor is reduced to a desired extent.

In some embodiments, the IRM compound is administered to a subject over a period that can range from about two weeks to about two years, although some embodiments of the invention may be performed by administering the IRM compound for a period outside this range. In some embodiments, the IRM compound may be administered over a period of from about one month to about six months, for example, for a period of about sixteen weeks.

The methods of the present invention may be performed on any suitable subject. Suitable subjects include but are not limited to animals such as but not limited to humans, non-human primates, rodents, dogs, cats, horses, pigs, sheep, goats, or cows.

### EXAMPLES

The following examples have been selected merely to further illustrate features, advantages, and other details of the invention. It is to be expressly understood, however, that while the examples serve this purpose, the particular materials and amounts used as well as other conditions and details are not to be construed in a matter that would unduly limit the scope of this invention.

Compounds

The compounds used in the examples are shown in Table 1.

TABLE 1

| Compound | Chemical Name | cLogP | Reference |
|---|---|---|---|
| 1 | 1-hydroxy-1-(1-ethylpropyl)-3- | 5.43 | U.S. Pat. No. |
| | (3-phenoxyphenyl)urea | | 5,612,377 |
| | | | Compound 77 |
| 2 | 3-(4-butoxyphenyl)-1-hydroxy- | 5.06 | U.S. Pat. No. |
| | 1-pentylurea | | 5,612,377^{#} |
| 3 | 1-hydroxy-1-(3-pyrrol-1- | 2.84 | U.S. Pat. No. |
| | ylpropyl)-3-phenylurea | | 5,612,377^{#} |
| 4 | 1-hydroxy-1-pentyl-3-(3,4,5- | 2.86 | U.S. Pat. No. |
| | trimethoxyphenyl)urea | | 5,612,377^{#} |
| 5 | 4,4′-methylenebis[1-hydroxy-1- | 6.60 | U.S. Pat. No. |
| | (1-ethylpropyl)-3-phenylurea | | 6,121,323 |
| | | | Example 12 |
| 6 | 1-hydroxy-3-(4-phenoxyphenyl)- | 5.66 | U.S. Pat. No. |
| | 1-pentylurea | | 5,612,377^{#} |
| 7 | 1-hydroxy-1-(1-methylethyl)-3- | 2.84 | U.S. Pat. No. |
| | [4-(methylthio)phenyl]urea | | 5,612,377 |
| | | | Compound 15 |
| 8 | 1-hydroxy-1-(1-ethylpropyl)-3- | 3.34 | U.S. Pat. No. |
| | phenylurea | | 5,612,377 |
| | | | Compound 42 |
| 9 | 1-hydroxy-1-methyl-3-[4- | 2.00 | U.S. Pat. No. |
| | (methylthio)phenyl]urea | | 5,612,377 |
| | | | Compound 1 |
| IRM1 | 4-amino-α,α,2-trimethyl-1H- | | U.S. Pat. No. |
| | imidazo[4,5-c]quinoline-1- | | 5,266,575 |
| | ethanol | | Example C1 |
| IRM2 | 4-amino-α,α-dimethyl-2- | | U.S. Pat. No. |
| | ethoxymethyl-1H-imidazo[4,5- | | 5,389,640 |
| | c]quinolin-1-ethanol | | Example 99 |

^{#}Compound is not explicitly exemplified, but can be prepared using the synthetic routes disclosed in the cited reference.

### Example 1

Compound 1 and Compound 2 were obtained from 3M Pharmaceuticals (St. Paul, Minn.). Each compound was dissolved in 85% EtOH/15% distilled water (unless otherwise indicated, all percentages provided herein are weight/weight with respect to the total formulation).

Eight-week old female A/J mice (The Jackson Laboratories, Bar Harbor, Me.) were divided into seven groups (n=12): Control, Placebo, Low Dose Compound 1 (1L), High Dose Compound 1 (1H), Low Dose Compound 2 (2L), High Dose Compound 2 (2H), and Zileuton. Mice in each group except for those in the Control group received three doses of the carcinogen benzo(a)pyrene (B(a)P, Aldrich Chemical Co., Milwaukee, Wis.) at 2 mg/20 g body weight, provided in 0.2 mL NF grade cottonseed oil (Croda USA, Parsippany, N.J.) via gavage. Doses of B(a)P were administered on Days 1, 4, and 7.

On Day 14, seven days after the final dose of B(a)P, the mice were dosed with test compound (Zileuton, Compound 1, or Compound 2) for sixteen weeks. Mice in the Zileuton group received an average oral dose of 245 mg/kg, provided in the diet ad libitum. Mice receiving aerosol doses of Compound 1, Compound 2, or placebo were dosed daily, five days per week.

Aerosol dosing was performed using a thirty-six port nose-only inhalation chamber (In-Tox Products, Moriarty, N.Mex.). Test atmospheres containing either Compound 1, Compound 2, or placebo (vehicle only) were generated using a Lovelace Aerosol Nebulizer and Diluter (In-Tox Products, Moriarty, N.Mex.). The aerosol concentration of Compound 2 was determined by HPLC analysis of a glass filter (47 mm, Pall Corp., Ann Arbor, Mich.) sampler attached to one of the inhalation ports. Retention time of the test compound was 4.06 minutes (245 nm, Mobile Phase 60:40 acetonitrile:water, isocratic elution). Aerosol particle size distribution was monitored using a Model 3321 AERODYNAMIC PARTICLE SIZER Spectrometer (APS TSI, Inc., Shoreview, Minn.). Particle size was determined for each drug group daily. The mass median aerodynamic diameter (MMAD) for all aerosols was maintained between 0.96 μm and 1.24 μm.

Low dose groups were exposed to the test atmospheres for ten minutes. High does groups were exposed to test atmospheres for twenty minutes. Aerosol concentration for each of Compound 1 and Compound 2 was determined to be 0.011 mg/L of air, corresponding to a calculated low dose of 220 μg/kg and a calculated high dose of 425 μg/kg.

After the dosing period, the animals were sacrificed and adenomas were visually assessed as described in Wexler, H., “Accurate Identification of Experimental Pulmonary Metastases,” J. Natl. Cancer Inst., 36:641-645 (1966). Average adenomas counts for mice in each group are shown in FIG. 1. Adenoma counts were similar for mice in groups 1H and 2H, the groups receiving high doses of Compound 1 and Compound 2, respectively.

### Example 2

Compounds were prepared in DMSO to a final concentration of 5 mM and assessed for inhibition of LTC₄ by radioimmuno assay (RIA). Sensitivity of the assay was 195 picograms/mL (pg/mL). LTC₄ was purchased from Biomol International, L.P., Plymouth Meeting, Pa.; ³H-LTC₄ was purchased from PerkinElmer Life and Analytical Sciences, Inc., Boston, Mass.; and antibody to LTC4 was purchased from Advanced Magnetics, Inc., Cambridge, Mass. The sensitivity of this assay was 195 pg/mL.

Sustained 5-lipoxygenase Inhibition in Vitro

Resident mouse peritoneal macrophages were obtained from male CD-1 mice (Charles River Laboratories, Inc. Wilmington, Mass.), 20 g-25 g, by lavage of the peritoneal cavity using 5 mL of Medium 199 containing 20 μg/mL gentamycin, 2.175 mg/mL sodium bicarbonate, 1% fetal calf serum and 20 u/mL heparin (unless otherwise indicated, all cell culture components obtained from Invitrogen Corp., Grand Island, N.Y.). The retrieved lavage medium was added to tissue culture dishes and incubated for two hours at 37° C. in a humidified atmosphere containing 5% CO₂. Following macrophage enrichment by adherence, the culture medium was removed and the resultant cell layer was washed twice with PBS, the medium (with heparin) was replaced with 1 mL of medium (without heparin), and the cells were then incubated overnight as described above. The following morning the medium was removed and the macrophages were washed twice with 2 mL PBS. One mL of fresh medium, devoid of serum, was then added.

Test compounds were added to the macrophage cultures to achieve a final concentration of 10 μM. The final DMSO concentration was 0.1% in for each test compound. Blank and control cultures were treated with DMSO only. After 30 minutes incubation, the macrophages were washed twice with 2 mL PBS. The medium was replaced with 1 mL Medium 199, and then treated in either of two ways: (a) challenged with zymosan (50 μg/mL), incubated one hour, the medium removed and assayed for LTC₄ by RIA, or (b) incubated for an additional six hours, this medium was removed, the cells were washed in PBS, 1 mL of fresh medium was added, and then treated as in (a). This treatment described in (b) was to allow for a washout period for the test compound.

For the evaluation of test compounds using metered dose inhalers, dry compounds were dissolved in ethanol to a concentration of 2.33 mg/mL. 2.54 mL of this solution was added to a 20 g glass metered dose inhaler (MDI) vial and fitted with a continuous valve. The vials were cooled on dry ice, filled with 18 g of HFA 134a (DYMEL, E. I. Du Pont de Nemours and Co., Corpus Christi, Tex.), returned to dry ice, and the continuous valve was replaced with an intermittent valve that delivered 25 μL per activation. A single activation of a vial prepared as described delivered 20 μg of compound per activation. For experiments requiring different mass of compound per activation, the initial ethanol solution concentration was adjusted as necessary.

Results are shown in Table 2.

A23187-Stimulated LTB₄ in Rat Lung in Situ

A. In Situ Dosing

Male Sprague-Dawley rats (Charles River Laboratories, Inc. Wilmington, Mass.) weighing 250-300 grams were anesthetized with methoxyflurane in a closed container, the peritoneal cavity was exposed, and the animals were exsanguinated. The pleural cavity was exposed and the lung was removed with both the trachea and heart attached. The trachea was fitted with a cannula, secured with a ligature, and the distal end of the cannula was extended through a small hole in a #6 rubber stopper allowing for a tight seal. The lung, secured to the cannula and stopper, was then suspended in a 125 mL side arm Erlenmeyer flask filled to 100 mL with PBS warmed to 37° C. As such, access to the airways of the lung could be achieved through the cannula extending through the stopper to the outside of the flask. The flask was then placed in a 37° C. water bath.

An MDI, prepared as described above, containing test compound was fired with a single activation through the cannula and into the airways of the lung. The MDI were made to deliver 20 μg/shot, thus providing a dose of from about 0.06 mg/kg to about 0.08 mg/kg. Control lungs were similarly treated with an MDI containing propellant and co-solvent. The lungs were then incubated for 5 minutes at 37° C. Following this preincubation, 25 μL of 10 mM A23187 (Sigma Chemical Co., St. Louis, Mo.) was fired into the airways and the lung was incubated for an additional 10 minutes. Control lungs again were treated with propellant and co-solvent, only.

The lungs were then lavaged with 5 mL of ice cold PBS containing 1 mM EDTA. The recovered lavage fluid was centrifuged for 10 minutes at 150×g to sediment cells and LTB₄ was quantified in the supernatant by specific RIA. The sensitivity of this assay was 155 pg/mL. Results are shown in Table 2.

B. In Vivo Dosing

Rats were anesthetized with methoxyflurane and fitted with a 16 gauge tracheal cannula through the mouth. The distal end of the cannula was fitted to a 12-inch length of 3/8 inch tubing. The tubing was, in turn, connected to the outlet of a 50 mL spacer unit. The spacer unit was kept under positive pressure by connecting to a reciprocating Harvard syringe pump (Harvard Apparatus, Inc., Holliston, Mass.) set to 60 strokes per minute and 5 mL per stroke. The top of the spacer was fitted with an actuator bearing a 0.015-inch aperture. With the rat under anesthesia and the tracheal cannula in place, the rat's respiratory rate synchronized with the period of the pump. Thus, an MDI could be fired into the spacer and delivered under positive pressure to the lungs of the anesthetized rat. Test compounds were delivered within a particle size range of 1-4 microns as determined using an AERODYNAMIC PARTICLE SIZER (Model 3321, TSI, Inc., Shoreview, Minn.).

Following dosing with this procedure the rat regained consciousness and remained conscious until secondary anesthesia, exsanguination, excision, and A23187 challenge as described above.

Quantifying of Compound Deposition in the Lung

Lungs from animals dosed by inhalation in vivo were obtained as described above and placed in ice cold PBS and processed immediately or were flash frozen and stored at −70° C. until processing. All tissues and liquids used in processing were maintained on ice throughout the procedure. The lungs were initially minced with a razor bladed, a known quantity of an appropriate internal standard was added, and the lung was homogenized in 10 mL of PBS using a POLYTRON homogenizer (Brinkmann Instruments, Inc., Westbury, N.Y.). The resulting homogenate was brought to 80% methanol (MeOH), by volume, homogenized further, and then stored overnight at −20° C.

The following day, the samples were centrifuged at 250×g for 30 minutes. The supernatant was retrieved and PBS was added to reduce the MeOH concentration to 60%. The supernatant was then added to a 20 mL C18 solid phase extraction column (Supelco, Sigma-Aldrich Co., St. Louis, Mo.) pre-equilibrated with MeOH, H₂O, and then 60% MeOH. The column was washed with 10 mL of 60% MeOH and the compounds were then eluted in 10 mL of MeOH. The MeOH was removed by vacuum desiccation and the residue was resuspended in 0.25 mL of acetonitrile.

The internal standard and the test compounds were separated on a 5 μm, 15 cm×4.6 mm Supelcosil LC—8 reverse phase column (Supelco, Sigma-Aldrich Co.) utilizing a 20 minute linear gradient of 0.1% H₃PO₄ to acetonitrile containing 0.1% H₃PO₄. Recovery of test compounds ranged from 85 to 94%.

TABLE 2

| | Sustained Inhibition, | Inhibition, | |
|---|---|---|---|
| Compound | 10 μM Initial | 20 μg in situ | cLogP |
| 9 | 0 | NT | 2.00 |
| 8 | 12 | 72 | 3.34 |
| 7 | 18 | NT | 2.84 |
| 3 | 3 | 84 | 2.84 |
| 1 | 100 | 100 | 5.43 |
| 5 | 86 | 66 | 6.60 |
| 6 | 100 | 88 | 5.66 |
| 4 | 24 | 84 | 2.86 |
| Zileuton | 28 | 42 | 2.48 |

### Example 3

Resident mouse peritoneal macrophages were added to 24 well culture dishes, 2×10⁶ cells/well, in 1 mL of medium containing 0.1 mM aspirin to inhibit irreversibly COX-I and to allow the macrophages to adhere to the culture dish. After 2 hours, the medium was removed and the cells were washed twice with 2 mL of PBS. Fresh medium (1 mL) containing the indicated amount of IRM1 was added to each well. The cells were then incubated overnight. The following morning, the medium was removed and the cells were washed twice with 2 mL of PBS. One mL of fresh medium was added to the cells and they were then challenged with 50 μg/mL of zymosan. After 2 hours, the medium was removed and zymosan-induced LTC₄ and PGE2 was quantified by RIA. Results are shown in FIG. 2.

### Example 4

A lung metastatic mouse model was used to determine the anti-tumor activity of a 5-LO inhibitor (zileuton) in combination with an IRM. 12-18 week old CDF1 female mice (Charles River Laboratories, Inc.) weighing 20-22 grams were injected in the tail intravenously with 5×10⁵ MC-26 murine colon carcinoma cells.

IRM2 was prepared in a 0.03M citrate buffered saline solution, pH 4.0. Zileuton was prepared in a 10% ethanol in water solution. At four hours and 24 hours after tumor challenge, mice were injected intra-peritoneally with zileuton and IRM2 as indicated in Table 3. One group of mice (Vehicle) was untreated. Fourteen days after tumor challenge, mice were sacrificed and lungs were removed and weighed. The tumor loads expressed as lung weights are shown in Table 3.

TABLE 3

| | Mean lung weight |
|---|---|
| Treatment | (g) |
| Vehicle (n = 5) | 0.463 |
| Zileuton (0.5 mg/kg) + IRM2 (0.01 mg/kg) (n = 5) | 0.497 |
| Zileuton (0.5 mg/kg) + IRM2 (1.0 mg/kg) (n = 4) | 0.284 |
| Zileuton (5.0 mg/kg) + IRM2 (0.01 mg/kg) (n = 5) | 0.387 |
| Zileuton (5.0 mg/kg) + IRM2 (1.0 mg/kg) (n = 5) | 0.255 |

The complete disclosures of the patents, patent documents and publications cited herein are incorporated by reference in their entirety as if each were individually incorporated. In case of conflict, the present specification, including definitions, shall control.

Various modifications and alterations to this invention will become apparent to those skilled in the art without departing from the scope and spirit of this invention. Illustrative embodiments and examples are provided as examples only and are not intended to limit the scope of the present invention. The scope of the invention is limited only by the claims set forth as follows.

1. A method for treating lung cancer in a subject, the method comprising: administering to the subject an inhalable formulation that comprises a 5-lipoxygenase inhibitor having a cLogP of at least about 4.0 in an amount effective for treating lung cancer.

2. The method of claim 1 wherein the 5-lipoxygenase inhibitor comprises a hydroxyurea.

3. The method of claim 1 wherein the 5-lipoxygenase inhibitor has a cLogP of at least 4.0.

4. The method of claim 1 further comprising administering to the subject an effective amount of an IRM compound.

5. The method of claim 4 wherein an effective amount of an IRM compound is an amount effective to further inhibit 5-lipoxygenase.

6. The method of claim 4 wherein an effective amount of an IRM compound is an amount effective to stimulate an immune response.

7. The method of claim 4 further comprising administering to the subject a tumor antigen in an amount effective, in combination with the IRM compound, to stimulate an immune response against the antigen.

8. The use of a 5-lipoxygenase inhibitor having a cLogP of at least 4.0 for the manufacture of an inhalable pharmaceutical composition for treating lung cancer.

9. A pharmaceutical combination comprising:
a 5-lipoxygenase inhibitor in an amount effective to inhibit 5-lipoxygenase; and
an effective amount of an IRM compound.

10. The pharmaceutical combination of claim 9 wherein an effective amount of an IRM compound is an amount effective to further inhibit 5-lipoxygenase.

11. The pharmaceutical combination of claim 9 wherein an effective amount of an IRM compound is an amount effective to stimulate an immune response.

12. The pharmaceutical combination of claim 9 further comprising a tumor antigen in an amount effective, in combination with the IRM compound, to stimulate an immune response against the antigen.

13. The pharmaceutical combination of claim 9 wherein the combination is provided in an inhalable formulation.

14. The pharmaceutical combination of claim 9 wherein the combination is provided in a plurality of formulations.

15. The pharmaceutical combination of claim 9 wherein the IRM compound comprises an imidazoquinoline amine, a tetrahydroimidazoquinoline amine, an imidazopyridine amine, a 1,2-bridged imidazoquinoline amine, a 6,7-fused cycloalkylimidazopyridine amine, an imidazonaphthyridine amine, a tetrahydroimidazonaphthyridine amine, an oxazoloquinoline amine, a thiazoloquinoline amine, an oxazolopyridine amine, a thiazolopyridine amine, an oxazolonaphthyridine amine, a thiazolonaphthyridine amine, a pyrazolopyridine amine, a pyrazoloquinoline amine, a tetrahydropyrazoloquinoline amine, a pyrazolonaphthyridine amine, or a tetrahydropyrazolonaphthyridine amine.

16. The use of an IRM compound and a 5-lipoxygenase inhibitor for the manufacture of a pharmaceutical composition for treating lung cancer.

17. A pharmaceutical composition comprising a 5-LO inhibitor having a cLogP of at least 4.0 in an amount effective to inhibit 5-lipoxygenase in an inhalable formulation.
